(19) United States
(12) Patent Application Publication
(10) Pub. No.: US 2022/0192865 A1
(43) Pub. Date: Jun. 23, 2022

(54) Targeted Temperature Management Systems, Pads, and Methods Thereof

(71) Applicant: C. R. Bard, Inc., Franklin Lakes, NJ (US)

(72) Inventors: James David Hughett, SR., Monroe, GA (US); Gabriel A. Johnston, Broomfield, CO (US); Madeline Stich, Thornton, CO (US); Sean E. Walker, Platteville, CO (US); Rebecca D. Salisbury, Lafayette, CO (US); Michael R. Hoglund, Windsor, CO (US); Karthik Ganesan, Longmont, CO (US); Tyghe E. Boone-Worthman, Brighton, CO (US)

(21) Appl. No.: 17/547,128

(22) Filed: Dec. 9, 2021

Related U.S. Application Data
(60) Provisional application No. 63/225,307, filed on Jul. 23, 2021.
(60) Provisional application No. 63/130,279, filed on Dec. 23, 2020.

(51) Int. Cl. A61F 7/00

(52) U.S. Cl. CPC A61F 7/0085; A61F 2007/0056

(57) ABSTRACT

Disclosed herein are systems, pads, and methods thereof for targeted temperature management. In an example, a pad for targeted temperature management can include a pad body, a conduit system, one or more inlets, and one or more outlets. The pad body can be of a conformable material configured to conform to a body of a patient on the pad. The conduit system can be disposed in the pad body or an overlayer configured for placement over the pad body. The conduit system can include one or more conduits configured to convey a fluid through the pad body or the overlayer. The one-or-more inlets can be configured for charging the conduit system with the fluid, while the one-or-more outlets can be configured for discharging the fluid from the conduit system. Methods of the systems and pads can include methods of use. In another example, a deformable pad can be configured for receiving a neonatal patient.

### PRIORITY

This application claims the benefit of priority to U.S. Provisional Patent Application No. 63/130,279, filed Dec. 23, 2020, and to U.S. Provisional Patent Application No. 63/225,307, filed Jul. 23, 2021, each of which is incorporated by reference in its entirety into this application.

### BACKGROUND

Existing solutions for target temperature management (“TTM”) require adhesive pads adhered onto patients or wraps with fastenable straps wrapped around the patients in order to maintain sufficient contact between the pads or wraps and the patients. Not only can such pads or wraps cause skin irritation about the edges thereof, but the varying sizes of the pads and wraps can cause inventory issues, which can ultimately result in the wrong size of pad or wrap being chosen for a patient. In addition, clinicians often require open access to the patients for medical procedures, but the existing pads and wraps can hinder the required access.

In addition to the foregoing, neonatal patients are prone to rapid heat loss and consequent hypothermia because of a high surface area to volume ratio, which is even higher in low-birth-weight neonates. There are several mechanisms for heat loss including evaporation, convection, conduction, and radiation. Despite their compensatory mechanisms, neonates, particularly low-birth-weight infants, have limited capacity to thermoregulate and are prone to decreased core temperature. Prolonged, unrecognized cold stress can divert calories to produce heat, impairing growth. In some instances, a neonatal reaction to cold stress increases the metabolic rate and oxygen consumption two- to three-fold. Cold stress can also result in tissue hypoxia and neurologic damage. Activation of glycogen stores can cause transient hyperglycemia. Persistent hypothermia can result in hypoglycemia and metabolic acidosis and increases the risk of late-onset sepsis and mortality.

Neonatal hyperthermia, while not as common as hypothermia, can result from maternal fever, maternal epidural anesthesia, excessive bundling or swaddling, or an infection. Symptoms of hyperthermia can include high heart rate, rapid breathing, perspiration, dehydration, poor feeding. Hence, it is imperative to maintain a healthy body temperature for neonatal patients.

TTM systems, pads, and methods thereof, including those for neonates, are disclosed herein to address the foregoing. Indeed, the TTM systems, pads, and methods thereof aid in the regulation of the body temperature of patients including neonatal patients.

### SUMMARY

Disclosed herein is a pad for TTM including, in some embodiments, a pad body, a conduit system, one or more inlets, and one or more outlets. The pad body is of a conformable material configured to conform to a body of a patient on the pad. The conduit system is disposed in the pad body or an overlayer configured for placement over the pad body. The conduit system includes one or more conduits configured to convey a fluid through the pad body or the overlayer. The one-or-more inlets are configured for charging the conduit system with the fluid. The one-or-more outlets are configured for discharging the fluid from the conduit system.

In some embodiments, the one-or-more conduits include a primary conduit and one or more expansion conduits. Each successive conduit of the one-or-more expansion conduits is configured to accommodate a successively larger patient placed on the pad for TTM.

In some embodiments, the primary conduit is located in a head-end portion of the pad body or the overlayer. Each successive conduit of the one-or-more expansion conduits is located further toward a foot-end portion of the pad body or the overlayer.

In some embodiments, each conduit of the one-or-more conduits is separated from other conduits of the one-or-more conduits. Each conduit of the one-or-more conduits includes a dedicated inlet of the one-or-more inlets and a dedicated outlet of the one-or-more outlets.

In some embodiments, the one-or-more conduits include a supply conduit configured to supply the primary conduit and each successive conduit of the one-or-more expansion conduits in parallel with the fluid. The supply conduit includes a dedicated inlet of the one-or-more inlets and a dedicated outlet of the one-or-more outlets.

In some embodiments, the primary conduit and each successive conduit of the one-or-more expansion conduits includes a hand-actuated valve having a closed state and an open state.

In some embodiments, the primary conduit and each successive conduit of the one-or-more expansion conduits includes a pressure-activated valve having a closed state and an open state. The valve is configured to assume the open state by pressure applied thereto by the patient when the patient is placed or lies down on the pad over the valve.

In some embodiments, the primary conduit and each successive conduit of the one-or-more expansion conduits includes an electrically activated valve having a closed state and an open state. The valve is configured to assume the open state upon sensing the patient with an associated sensor when the patient is placed or lies down on the pad over the sensor.

In some embodiments, the sensor is a tactile sensor or thermal photodetector configured to sense the patient physically touching the sensor when the patient is placed or lies down on the pad over the sensor.

In some embodiments, the pad is configured for placement over a mattress of a hospital bed.

Also disclosed is a system for TTM including, in some embodiments, a control module and a pad. The control module includes a chiller evaporator and a hydraulic system. The chiller evaporator is configured for chilling a fluid to produce a chilled fluid. The hydraulic system includes one or more outlets configured for discharging the chilled fluid from the hydraulic system and one or more inlets configured for charging the hydraulic system with the fluid to continue to produce the chilled fluid. The pad includes a pad body, a conduit system, one or more inlets, and one or more outlets. The pad body is of a conformable material configured to conform to a body of a patient on the pad. The conduit system is disposed in the pad body or an overlayer configured for placement over the pad body. The conduit system includes one or more conduits configured to convey the chilled fluid through the pad body or the overlayer. The one-or-more inlets are configured for charging the conduit system with the chilled fluid. The one-or-more outlets are configured for discharging the fluid from the conduit system.

In some embodiments, the one-or-more conduits include a primary conduit and one or more expansion conduits. Each successive conduit of the one-or-more expansion conduits is configured to accommodate a successively larger patient placed on the pad for TTM.

In some embodiments, the primary conduit is located in a head-end portion of the pad body or the overlayer. Each successive conduit of the one-or-more expansion conduits is located further toward a foot-end portion of the pad body or the overlayer.

In some embodiments, each conduit of the one-or-more conduits is separated from other conduits of the one-or-more conduits. Each conduit of the one-or-more conduits includes a dedicated inlet of the one-or-more inlets of the pad and a dedicated outlet of the one-or-more outlets of the pad.

In some embodiments, the one-or-more conduits include a supply conduit configured to supply the primary conduit and each successive conduit of the one-or-more expansion conduits in parallel with the fluid. The supply conduit includes a dedicated inlet of the one-or-more inlets of the pad and a dedicated outlet of the one-or-more outlets of the pad.

In some embodiments, the primary conduit and each successive conduit of the one-or-more expansion conduits includes a hand-actuated valve having a closed state and an open state.

In some embodiments, the primary conduit and each successive conduit of the one-or-more expansion conduits includes a pressure-activated valve having a closed state and an open state. The valve is configured to assume the open state by pressure applied thereto by the patient when the patient is placed or lies down on the pad over the valve.

In some embodiments, the primary conduit and each successive conduit of the one-or-more expansion conduits includes an electrically activated valve having a closed state and an open state. The valve is configured to assume the open state upon sensing the patient with an associated sensor when the patient is placed or lies down on the pad over the sensor.

In some embodiments, the sensor is a tactile sensor or thermal photodetector configured to sense the patient physically touching the sensor when the patient is placed or lies down on the pad over the sensor.

In some embodiments, the pad is configured as a mattress of a hospital bed.

Also disclosed is a system including, in some embodiments, a neonatal bed and a thermal energy exchange mechanism. The neonatal bed includes a deformable pad configured for receiving a neonatal patient. The thermal energy exchange mechanism is coupled with the deformable pad. The thermal energy exchange mechanism is configured to extract thermal energy from the neonatal patient to cool the neonatal patient via a thermally conductive filling material of the deformable pad.

In some embodiments, the thermal energy exchange mechanism is configured to deliver thermal energy to the neonatal patient to warm the neonatal patient.

In some embodiments, the deformable pad is continuously deformable between a flat top surface and a contoured top surface. The contoured top surface includes a formed depression configured to receive the neonatal patient thereon.

In some embodiments, the filling material has viscosity between about 10,000 centipoise and 1,000,000 centipoise.

In some embodiments, the filling material has a thermal conductivity greater than about 0.1 Watt/meter-° C.

In some embodiments, the filling material includes embedded objects to enhance the thermal conductivity of the filling material.

In some embodiments, the filling material has a thermal conductivity greater than about 2.0 Watt/meter-° C.

In some embodiments, the thermal energy exchange mechanism includes a thermoelectric device (TED).

In some embodiments, the neonatal bed includes a tray thermally coupled between the TED and the deformable pad. The tray is configured to transfer thermal energy between the TED and the deformable pad.

In some embodiments, the tray is configured to laterally disperse thermal energy away from the TED to define a uniform temperature of the tray.

These and other features of the concepts provided herein will become more apparent to those of skill in the art in view of the accompanying drawings and following description, which describe particular embodiments of such concepts in greater detail.

### DRAWINGS

FIG. 1 illustrates a TTM system in accordance with some embodiments.

FIG. 2A illustrates an assembled TTM pad in accordance with some embodiments.

FIG. 2B illustrates a disassembled TTM pad in accordance with some embodiments.

FIG. 3 illustrates a conduit system of the pad of FIGS. 2A and 2B in accordance with some embodiments.

FIG. 4A illustrates a disassembled TTM pad in accordance with some embodiments.

FIG. 4B illustrates a pressure-sensitive valve of a conduit system of the pad of FIG. 4A in a closed state in accordance with some embodiments.

FIG. 4C illustrates a pressure-sensitive valve of a conduit system of the pad of FIG. 4A in an open state in accordance with some embodiments.

FIG. 5 illustrates the conduit system of the pad of FIG. 4A in accordance with some embodiments.

FIG. 6 illustrates a hydraulic system of a control module in accordance with some embodiments.

FIG. 7 illustrates a system for regulating a body temperature of a neonatal patient in accordance with some embodiments.

FIG. 8 illustrates a longitudinal cross section a first neonatal bed of the system of FIG. 1 in accordance with some embodiments.

FIG. 9 illustrates a detailed longitudinal cross section a second neonatal bed of the system of FIG. 1 in accordance with some embodiments.

FIG. 10 is a block diagram of a console of the system of FIG. 1 in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Furthermore, the terms “or” and “and/or” as used herein are to be interpreted as inclusive or meaning any one or any combination. As an example, “A, B, or C” or “A, B, and/or C” mean any of the following: A; B; C; A and B; A and C; B and C; or A, B and C. An exception to this definition occurs when a combination of elements, components, functions, steps, or acts are, in some way, inherently mutually exclusive.

The phrases “connected to,” “connected with,” “coupled to,” and “coupled with” refer to any form of interaction between two or more entities, including mechanical, electrical, magnetic, electromagnetic, fluid, signal, communicative (including wireless), and thermal interaction. Two components can be connected or coupled to each other even though they are not in direct contact with each other. For example, two components can be coupled to each other through an intermediate component.

Any methods disclosed herein include one or more steps, actions, or operations for performing the method. The method steps, actions, or operations can be interchanged with one another. In other words, unless a specific order of steps or actions is required for proper operation of the embodiment, the order or use of specific steps, actions, or operations can be modified. Moreover, sub-routines or only a portion of a method described herein can be a separate method within the scope of this disclosure. Stated otherwise, some methods can include only a portion of the steps described in a more detailed method.

Unless defined otherwise, all technical and scientific terms used herein have the same meaning as commonly understood by those of ordinary skill in the art.

As set forth above, existing solutions for TTM can cause skin irritation, inventory issues, and restrict patient access clinicians need for medical procedures. Not only is it important to maintain a healthy body temperature for all patients in need thereof, but neonatal patients present special circumstances under which it is imperative to maintain a healthy body temperature.

TTM systems, pads, and methods thereof, including those for neonates, are disclosed herein to address the foregoing. Indeed, the TTM systems, pads, and methods thereof aid in the regulation of the body temperature of patients including neonatal patients.

In an example a pad for targeted temperature management, the pad can include a pad body, a conduit system, one or more inlets, and one or more outlets. The pad body can be of a conformable material configured to conform to a body of a patient on the pad. The conduit system can be disposed in the pad body or an overlayer configured for placement over the pad body. The conduit system can include one or more conduits configured to convey a fluid through the pad body or the overlayer. The one-or-more inlets can be configured for charging the conduit system with the fluid, while the one-or-more outlets can be configured for discharging the fluid from the conduit system. Methods of the systems and pads can include methods of use. In another example, a deformable pad can be configured for receiving a neonatal patient.

In an example of a system for targeted temperature management, the system can include a neonatal bed configured for receiving a neonatal patient and a thermal energy exchange mechanism coupled with the neonatal bed. The neonatal bed can include a deformable pad that includes a thermally conductive filling material. The thermal energy exchange mechanism, which can include one or more thermoelectric devices, can be configured to exchange thermal energy with the neonatal patient to cool or warm the neonatal patient as needed.

The foregoing features of the TTM systems and pads, as well as other features of the TTM systems, pads, and methods thereof provided herein, will become more apparent to those of skill in the art in view of the accompanying drawings and following description, which describe particular embodiments of the foregoing in greater detail.

### TTM Systems

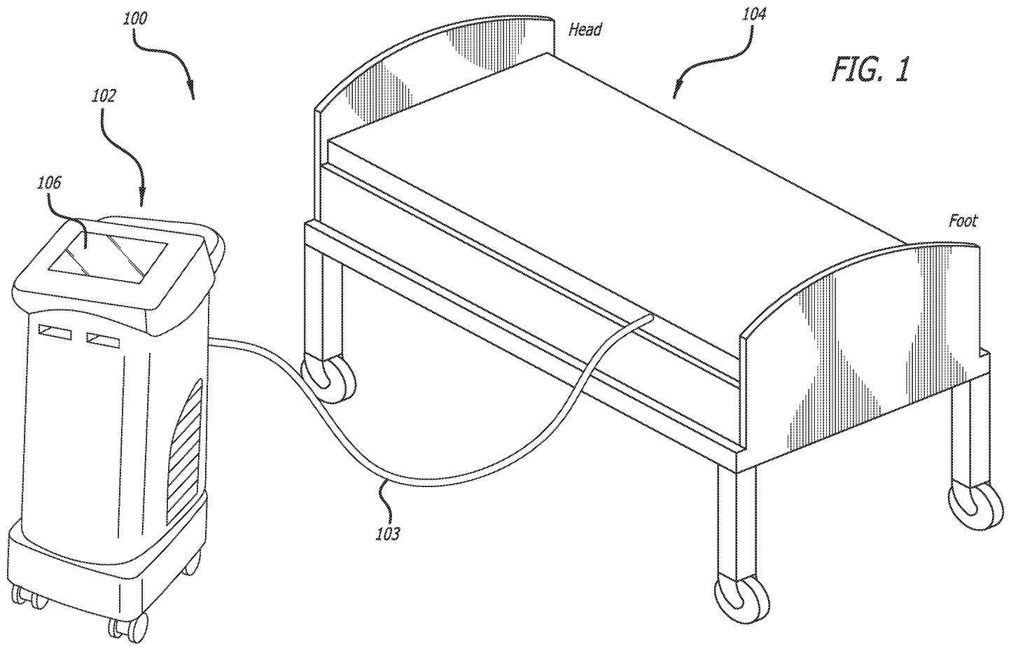

FIG. 1 illustrates a TTM system 100 in accordance with some embodiments.

As shown, the system 100 includes a TTM control module 102, a TTM pad 104, and one or more fluid conduits 103 therebetween. Description for the control module 102 is set forth immediately below. Description for the pad 104 is set forth in a following section.

The control module 102 includes a console 106 with an integrated display screen configured as a touchscreen for operating the control module 102. The console 106 includes one or more processors, primary and secondary memory, and instructions stored in the primary memory configured to instantiate one or more processes for TTM with the control module 102. For example, the one-or-more processes can include sensor logic for opening the one-or-more valves 160 as set forth below for the conduit system 144.

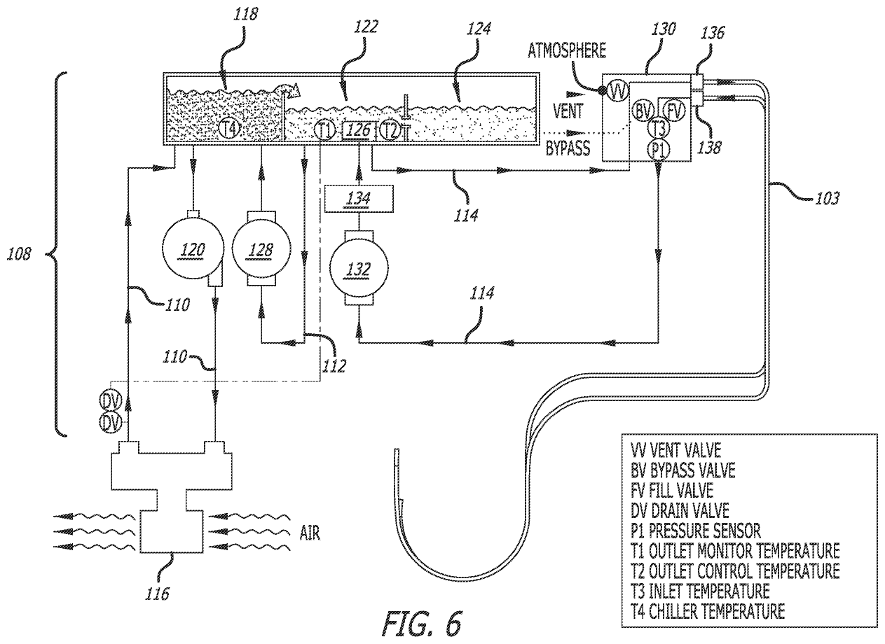

FIG. 6 illustrates a hydraulic system 108 of a control module in accordance with some embodiments.

The control module 102 also includes the hydraulic system 108, which includes a chiller circuit 110, a mixing circuit 112, and a circulating circuit 114.

The chiller circuit 110 is configured for chilling a fluid (e.g., water, ethylene glycol, a combination of water and ethylene glycol, etc.) to produce a chilled fluid, which chilled fluid, in turn, is for mixing with an optionally heated fluid to produce a supply fluid for TTM. The chiller circuit 110 includes a chiller evaporator 116 configured for the chilling of the fluid passing therethrough. The fluid for the chilling by the chiller evaporator 116 is provided by a chiller tank 118 using a chiller pump 120 of the chiller circuit 110.

The mixing circuit 112 is configured for mixing spillover of the chilled fluid from the chiller tank 118 with a mixed fluid in a mixing tank 122 of the mixing circuit 112. The mixing circuit 112 includes a heater 126 in the mixing tank 122 configured for heating the mixed fluid to produce a heated fluid if needed for mixing with the chilled fluid to provide a supply tank 124 of the circulating circuit 114 with a supply fluid of a desired temperature for TTM. The mixing circuit 112 includes a mixing pump 128 configured to pump the fluid from the mixing tank 122 into the chiller tank 118 for producing the chilled fluid and the spillover of the chilled fluid for the mixing tank 122.

The circulating circuit 114 is configured for circulating the supply fluid for TTM, which includes circulating the supply fluid provided by the manifold 130 through the pad 104 using a circulation pump 132 directly or indirectly governed by a flow meter 134 of the circulating circuit 114. The manifold 130 includes one or more outlets 136 configured for discharging the supply fluid (e.g., a chilled fluid) from the hydraulic system 108 and one or more inlets 138 configured for charging the hydraulic system 108 with return fluid from the pad 104 to continue to produce the supply fluid.

Add-on to the system 100 can include a temperature-determining means for determining a temperature of a patient's hands or feet. The temperature-determining means is useful in TTM because a patient's body will not shiver as much if the patient's hands and feet are warm, which will increase the effectiveness of therapeutic hypothermia.

Another add-on to the system 100 can include one or more massaging rollers in or under the pad 104. The massaging roller can be effective in increasing a patient's blood flow and decreasing bed sores.

### TTM Pads

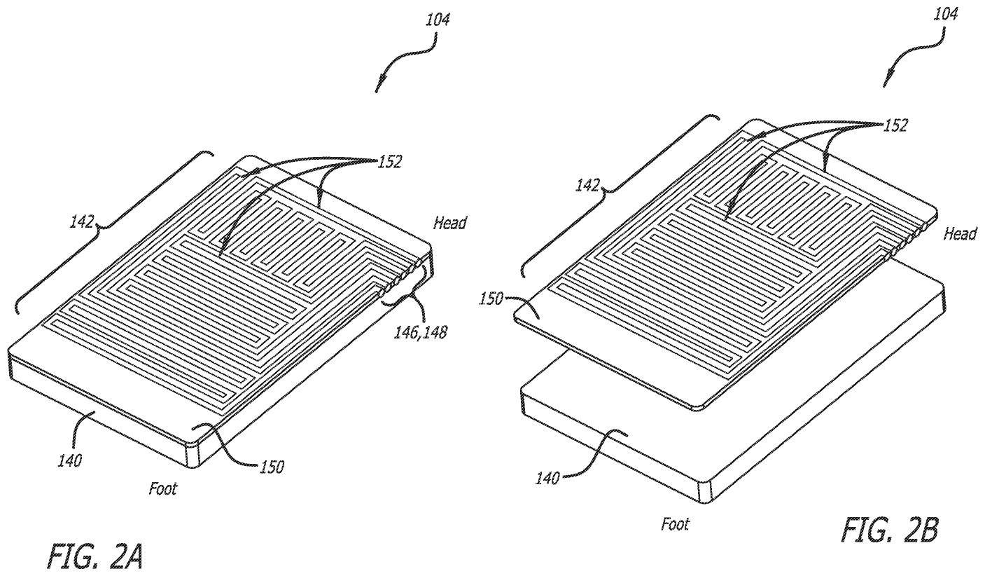

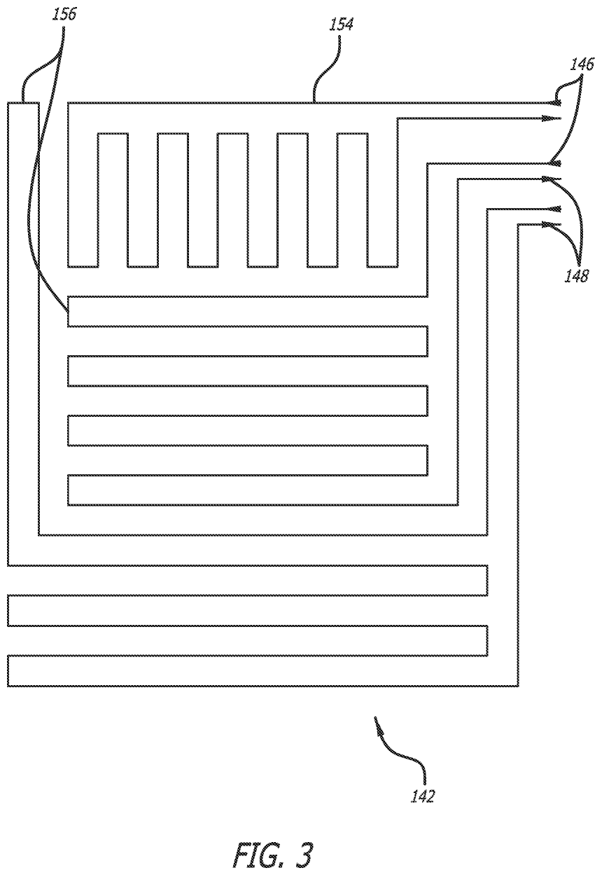

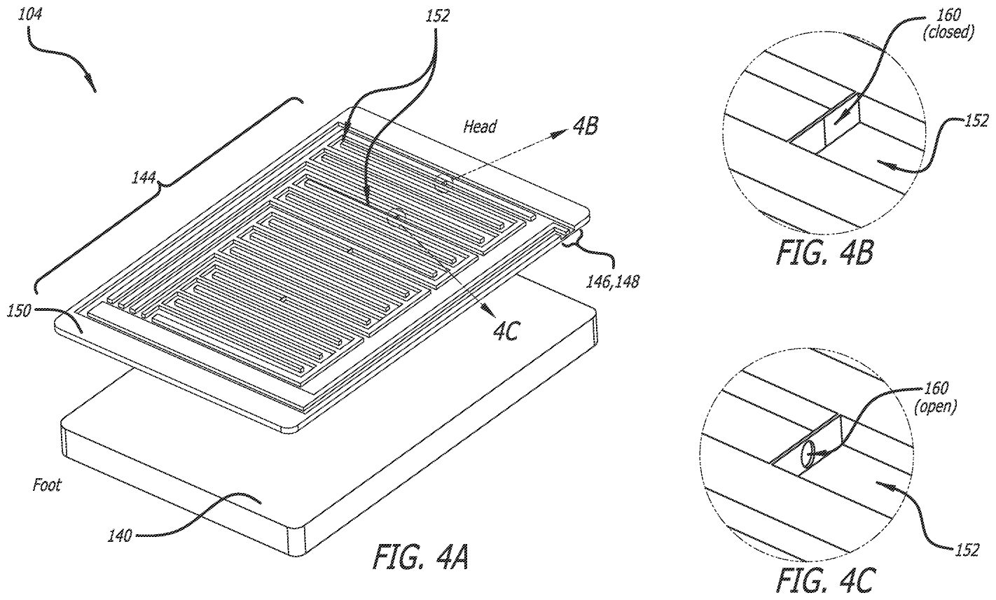

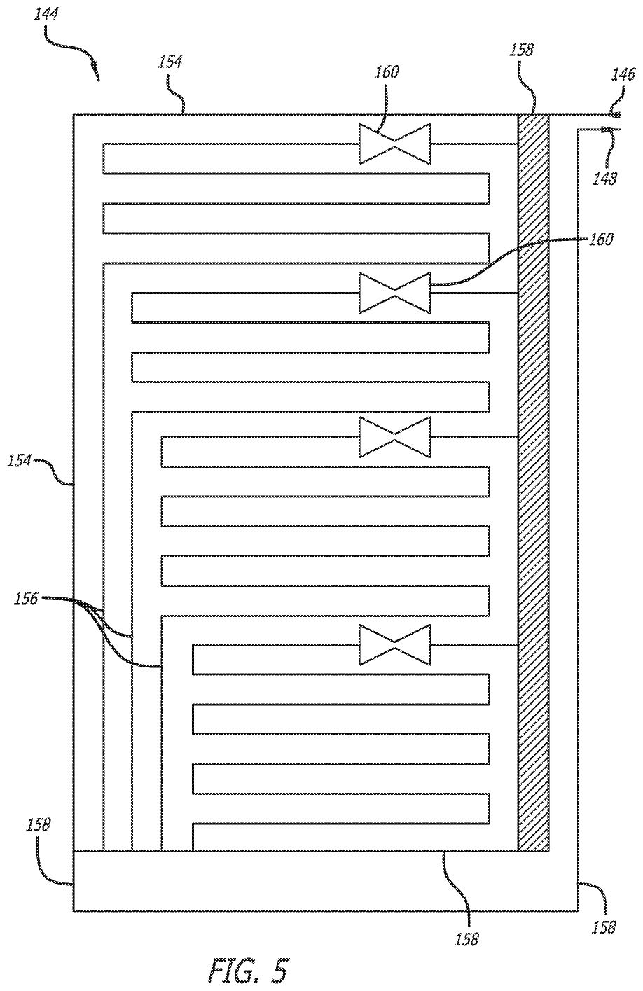

FIGS. 2A, 2B, and 4A illustrates the pad 104 in accordance with some embodiments. Indeed, FIG. 3 illustrates the pad 104 of FIGS. 2A and 2B with a first a conduit system, while FIG. 5 illustrates the pad 104 of FIG. 4A with a second conduit system of. FIGS. 4B and 4C illustrate a pressure-sensitive valve of the conduit system in a closed state and an open state, respectively.

As shown, the pad 104 includes a pad body 140, a conduit system 142 or 144, one or more inlets 146 configured for charging the conduit system 142 or 144 with the supply fluid (e.g., a chilled fluid) from the control module 102, and one or more outlets 148 configured for discharging the foregoing fluid from the conduit system 142 or 144. The pad 104 is either configured for placement over a mattress of a hospital bed as shown in FIG. 1 or as the mattress of a hospital bed. Regardless, the pad 104 can be easy to clean or fitted with a disposable, single-use cover for each new patient.

The pad body 140 is generally of a conformable material (e.g., a viscoelastic or low-resilience polyurethane foam) configured to conform to a body of a patient on the pad 104, thereby allowing for full-surface contact with a back side of the patient while allowing open access to the patient's internal organs or the like. While the pad body 140 of the conformable material is notable for its thermal conductivity, the pad body 140 need not be of a conformable material.

The conduit system 142 or 144 is either disposed in the pad body 140 or an overlayer 150 configured for placement over the pad body 140. Regardless, the conduit system 142 or 144 includes one or more conduits 152 configured to convey the supply fluid (e.g., a chilled fluid) from the control module 102 through the pad body 140 or the overlayer 150.

The one-or-more conduits 152 include a primary conduit 154 and one or more expansion conduits 156. Each successive conduit of the one-or-more expansion conduits 156 is configured to accommodate a successively larger (e.g., taller or longer) patient placed on the pad for TTM. Indeed, the primary conduit 154 is located in a head-end portion of the pad body 140 or the overlayer 150, and each successive conduit of the one-or-more expansion conduits 156 is located further toward a foot-end portion of the pad body 140 or the overlayer 150. (See, for example, FIGS. 3 and 5.) In addition, each successive conduit of the one-or-more expansion conduits 156 can be expanded further toward a side of the pad body 140 or the overlayer 150 opposite the one-or-more inlets 146 and the one-or-more outlets 148 to accommodate a successively larger (e.g., wider) patient placed on the pad for TTM. (See, for example, FIG. 3.)

As to the conduit system 142, each conduit of the one-or-more conduits 152 is separated from the other conduits of the one-or-more conduits 152. Each conduit of the one-or-more conduits 152 includes a dedicated inlet of the one-or-more inlets 146 and a dedicated outlet of the one-or-more outlets 148. Indeed, as shown in FIG. 3, the primary conduit 154 includes a first dedicated inlet and a first dedicated outlet, a first expansion conduit of the one-or-more expansion conduits 156 includes a second dedicated inlet and a second dedicated outlet, and a second expansion conduit of the one-or-more expansion conduits 156 includes a third dedicated inlet and a third dedicated outlet. Successively larger patients can be accommodated on the pad 104 with the conduit system 142 by opening the dedicated inlets and outlets of the of the one-or-more expansion conduits 156 as needed.

As to the conduit system 144, the conduit system 144 also includes a supply conduit 158 of the one-or-more conduits 152. The supply conduit 158 is configured to supply the primary conduit 154 and each successive conduit of the one-or-more expansion conduits 156 in parallel with the supply fluid (e.g., chilled fluid) from the control module 102. As such, the supply conduit 158 is configured with a dedicated inlet of the one-or-more inlets 146 and a dedicated outlet of the one-or-more outlets 148 to the exclusion of any other inlets or outlets of the conduit system 144. In contrast to the conduit system 142, successively larger patients can be accommodated on the pad 104 with the conduit system 144 by opening one or more valves 160 of the conduit system 144, each valve of the one-or-more valves 160 located in one conduit of the primary conduit 154 and the one-or-more expansion conduits 156.

Each valve of the one-or-more valves 160 can include a hand-actuated valve having a closed state and an open state.

Each valve of the one-or-more valves 160 can alternatively include a pressure-activated valve having a closed state and an open state as respectively shown in FIGS. 4B and 3C. The pressure-activated valve is configured to assume the open state by pressure applied thereto by the patient when the patient is placed or lies down on the pad 104 over the valve.

Each valve of the one-or-more valves 160 can alternatively include an electrically activated valve having a closed state and an open state. The electrically activated valve is configured to assume the open state upon sensing the patient with an associated sensor of the electrically activated valve when the patient is placed or lies down on the pad 104 over the sensor. The sensor can be a tactile sensor or a thermal photodetector configured to sense the patient physically touching the sensor when the patient is placed or lies down on the pad 104 over the sensor. The sensor can alternatively be a photoelectric photodetector configured to sense the patient blocking light to the sensor when the patient is placed or lies down on the pad 104 over the sensor.

Advantageously, the foregoing pads such as the pad 104 eliminates difficulties with respect to adhering pads to patients or wrapping the patients with wraps because the patients simply lie down on the pad 104 without rolling around for proper placement of the pads or wraps.

### TTM Systems and Pads for Neonates

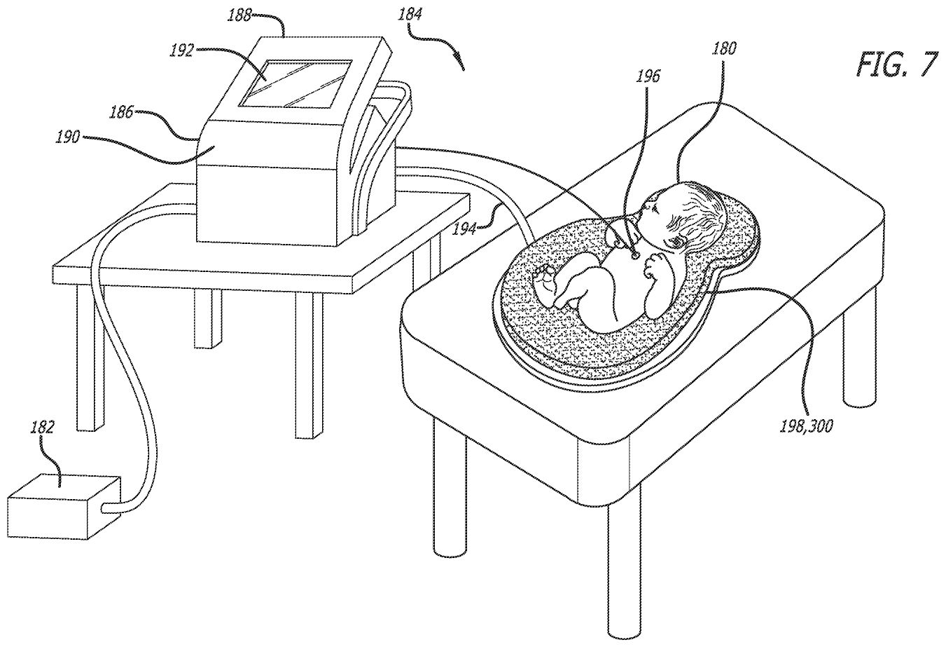

FIG. 7 illustrates an exemplary embodiment of a neonatal patient temperature regulation system 184. The system 184 generally includes a neonatal bed 198, 300 coupled with a control module 186 via an electrical cable 194. In use, a neonatal patient 180 is placed on the neonatal bed 198, 300 and the control module 186 defines a bed temperature to warm or cool the patient 180. In some embodiments, the system 184 can exchange thermal energy with the patient 180 to warm or cool the patient 180 in accordance a targeted temperature management (TTM) therapy. The neonatal bed 198, 300 can be sized and shaped to fit the patient 180 (i.e., a neonate) having a weight ranging from about 1.5 Kg and up to about 4.5 Kg.

The control module 186 includes console 190 disposed within a housing 188 and an operator interface 192 such as a graphical user interface (GUI). In some embodiments, the operator interface 192 can be disposed within the housing 188. The system 184 is coupled with an electrical power source 182 which provides power to the control module 186 including the console 190. In some embodiments, the power source 182 can be an electrical power system of a facility. In other embodiments, the power source 182 can be a portable power source such as a battery pack or a generator. The system 184 converts electrical power from the power source 182 into thermal energy exchange with the patient 180.

In use, the control module 186 can continually control the thermal energy exchange with the patient 180 to regulate the temperature of the patient 180. In some embodiments, the control module 186 can be coupled with a body temperature sensor 196. The body temperature sensor 196 is coupled to the patient 180 to indicate a core body temperature of the patient 180. In use, the control module 186 can acquire body temperature data from the body temperature sensor 196 and adjust the bed temperature to move the body temperature of the patient 180 toward a target body temperature. In other words, the control module 186 can regulate the bed temperature according to the target body temperature.

The operator interface 192 can facilitate operation of the system 184 by a clinician/operator. By way of the operator interface 192, the clinician can set a target body temperature of the patient, a target bed temperature, and other associated operating parameters of the system 184. The operator interface 192 can also display operating parameters of the system 184 such as the current body temperature and the current bed temperature, for example. In some embodiments, the operator interface 192 can be wirelessly coupled to the control module 186. For example, the operator interface 192 can comprise a software application running on an external device such as a network computer, a tablet, or a cell phone.

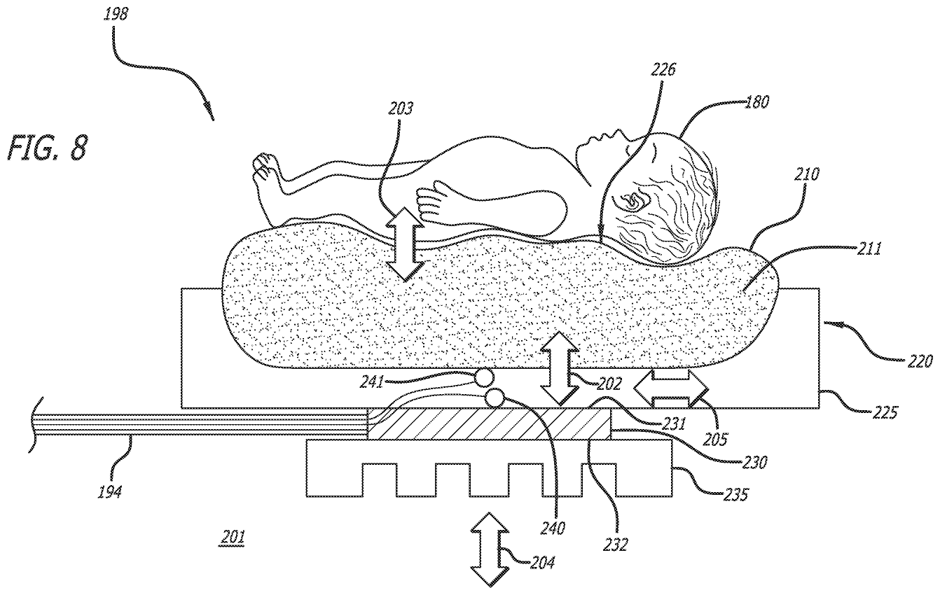

FIG. 8 illustrates a detailed cross-sectional side view of the neonatal bed 198. As shown, the neonatal bed 198 generally includes a pad 210 coupled with a thermal energy exchange mechanism 220. In general terms, the thermal energy exchange mechanism 220 exchanges thermal energy 202 with the pad 210 to define a temperature of the pad 210. The pad 210, in turn, exchanges thermal energy 203 with the patient 180. The thermal energy exchange mechanism 220 also exchanges thermal energy 204 with the environment 201.

The thermal energy exchange mechanism 220, by way of an exemplary embodiment, includes a tray 225 thermally coupled between the pad 210 and at least one thermoelectric device (TED) 230. The tray 225 is configured to conductively transfer thermal energy between the TED 230 and the pad 210. The tray 225 is also configured to conduct thermal energy 205 laterally across the tray 225 to define a uniform temperature of the tray 225. The tray 225 is formed of a thermally conductive material, such as aluminum, stainless steel, or a thermally conductive plastic, for example.

In some embodiments, the thermal energy exchange mechanism 220 can include multiple TEDs 230. The TEDs 230 can be controlled as one set via a single temperature sensor 240. In other embodiments, the TEDs 230 can be controlled as subsets, or individually via multiple temperature sensors 240.

In use, the pad 210 is placed on a top surface of the tray 225. In some embodiments, the tray 225 can include a contoured top surface 226. The contoured top surface 226 can include a depression to partially encapsulate the pad 210. Partial encapsulation can enhance the thermal energy exchange 202. The contoured top surface 226 can also be shaped to correlate with the size and contours of the patient 180. For example, if the patient 180 is generally oriented face up, then the contoured top surface 226 can follow the contours of a back side of the patient 180. The TED 230 can be coupled with the tray along a bottom side of the tray 225.

The TED 230 is configured to define a temperature difference between a top side 231 and a bottom side 232 of the TED 230 in accordance with a direct-current (DC) voltage supplied to the TED 230. The magnitude of the temperature difference between the top side 231 and the bottom side 232 is defined by the magnitude of the DC voltage supplied to the TED 230 and direction of the temperature difference is defined by the polarity of the DC voltage. The polarity of the DC voltage as defined by the control module 186 can define a temperature of the top side 231 that is hotter than the bottom side 232. Conversely, a reversed polarity of the DC voltage as defined by the control module 186 can define a temperature of the top side 231 that is colder than the bottom side 232. In use, the control module 186 supplies electrical energy to the TED 230 to establish the temperature difference between the top side 231 and the bottom side 232 and the temperature difference causes thermal energy exchange between the top side 231 and the tray 225.

By way of example, the control module 186 can supply electrical energy to the TED 230 to establish a temperature difference between the top side 231 and the bottom side 232 such that the temperature of the top side 231 (i.e., the tray temperature and, in turn, the pad temperature) is less than the temperature of the patient 180, and the temperature of the bottom side 232 is greater than the temperature of the environment 201. In this example, thermal energy exchange 203 is directed away from the patient 180 toward the TED 230 thereby cooling the patient 180. The thermal energy 204 then passes through the TED 230 from the top side 231 to the bottom side 232. Similarly, temperature of the bottom side 232 is greater than the environment 201 so that the thermal energy exchange 204 is directed away from the bottom side 232 of the TED 230 toward the environment 201. By way of summary, thermal energy is exchanged between the patient 180 and the environment 201 to cool the patient 180. In some embodiments, the thermal energy exchange mechanism 220 can include a thermal convection device 235 to enhance the thermal energy exchange 204 between the bottom side 232 of the TED 230 and the environment 201.

By way of an alternative example, the control module 186 can supply electrical energy to the TED 230 to establish a temperature difference between the top side 231 and the bottom side 232 such that the temperature of the top side 231 (i.e., the tray temperature and, in turn, the bed temperature) is greater than the temperature of the patient 180. In such an example, the temperature of the bottom side 232 can be greater than or less than the temperature of the environment 201. In this example, the thermal energy exchange 203 is directed toward the patient 180 thereby warming the patient 180.

The thermal exchange mechanism 220 includes the control temperature sensor 240 thermally coupled with the tray 225 and electrically coupled with the control module 186. The control temperature sensor 240 is used to regulate the temperature of the pad 210 as further described below. The thermal exchange mechanism 220 can also include a monitoring temperature sensor 241. The monitoring temperature sensor 241 is also thermally coupled with the tray 225 and electrically coupled with the control module 186. The monitoring temperature sensor 241 can be used to keep the patient 180 safe from extreme temperatures in the case of a failure of the temperature control process as further described below.

The pad 210 can be generally configured to facilitate thermal energy exchange between the patient 180 and the tray 225. More specifically, the pad 210 can contain a filling material 211 having thermal conduction properties to enhance thermal energy transfer through the pad 210. In some embodiments, the filling material 211 can have thermal conductivity greater than about 0.1 Watt/meter-° C., 0.3 Watt/meter-° C., or 0.6 Watt/meter-° C.

In some embodiments, the filling material 211 can include embedded components, which can include micro particles or powders, such as sand, for example. In some embodiments, the embedded components can include a material having a relatively high thermal conductivity, such as stainless steel or aluminum, for example, to further enhance the thermal conductivity of the filling material 211. Hence, in further embodiments, the filling material 211 can have a thermal conductivity greater than about 2.0 Watt/meter-° C., 10 Watt/meter-° C., or 50 Watt/meter-° C.

The pad 210 can also be generally configured to enhance a thermal coupling between the pad 210 and the patient 180 by maximizing direct contact of the pad 210 with the patient 180, and/or minimizing gaps between the pad 210 and the patient 180. To maximize direct contact, the filling material 211 can include moldable characteristics that allow the pad 210 to follow contours of the patient 180, which in some embodiments can be similar to a waterbed or a bean bag.

The pad 210 can be continuously deformable between a flat top surface and a contoured top surface, and the contoured top surface can include a depression configured to receive the patient 180 therein. The moldable characteristics of the pad 210 can also support the patient 180 in a defined position such on a back or side of the patient. More specifically the pad 210 can provide lateral support for the patient 180 so that the patient is maintained in defined position. For example, in use, the clinician can for a depression in the pad 210 having one of more lateral support walls. The clinician can place the patient in the depression so that the walls provide lateral support for the patient 180. After placing the patient 180 in the depression, the clinician can further deform the pad 210 to position the lateral support walls adjacent the patient 180. By way of a more specific example, the clinician can place the patient 180 in the depression on the patient's side. Thereafter, the clinician can deform the pad 210 to define a lateral wall against a back side of the patient's torso. The clinician can also deform the pad 210 to define a lateral wall against a front side the patient's torso so that the patient is prevented from rolling onto its back side or front side.

In some embodiments, the filling material 211 can include a gel or semi-solid material (which can be polymeric) having a viscosity between about 10,000 centipoise and 1,000,000 centipoise, 10,000 centipoise and 500,000 centipoise, or 50,000 centipoise and 300,000 centipoise. As such, in some embodiments, the filling material 211 can have a putty-like moldability characteristic. In some embodiments, the pad 210 can be comprised of viscoelastic polyurethane foam, or low-resistance polyurethane foam (LRPu).

In some embodiments, the filling material 211 can include a pseudoplastic (i.e., non-Newtonian) material. The pseudoplastic material can allow the clinician to deform the pad 210 to form contours in the pad 210 while preventing further deformation of the pad 210 via contact forces applied by the patient 180. As such, the clinician can form contours in the pad 210 in accordance with a desired position of the patient 180 and the pseudoplastic characteristic of the filling material 211 can maintain the patient 180 in the desired position.

The pad 210 can be separably coupled with the tray 225 to allow for cleaning/disinfection of the pad 210. In some, embodiments, the pad 210 can include a removable cover (not shown).

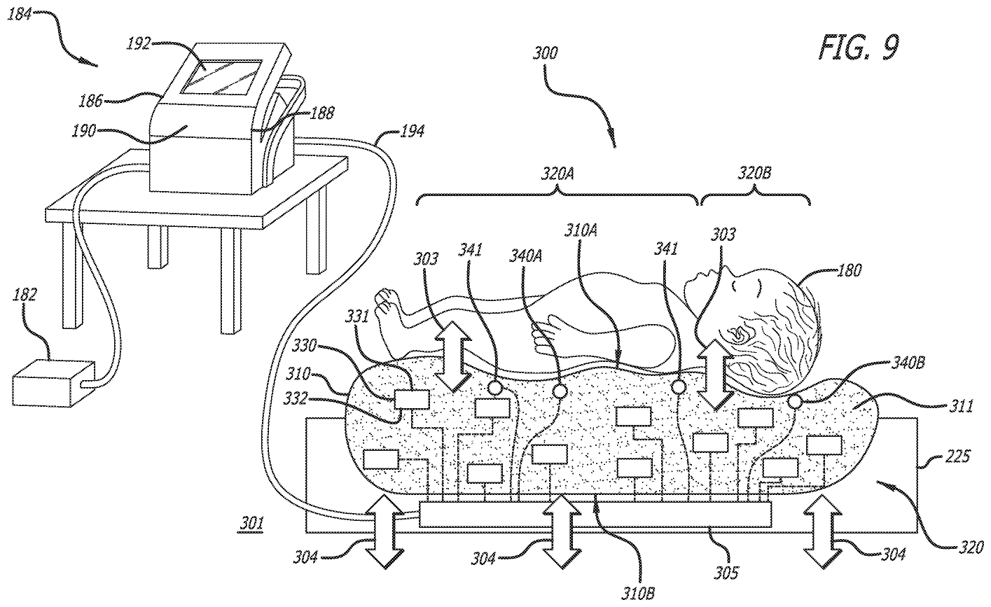

FIG. 9 illustrates a detailed cross-sectional view of a second embodiment of a neonatal bed as can be employed by system of FIG. 7, in accordance with some embodiments. As shown, the neonatal bed 300 generally includes a pad 310 coupled with a thermal energy exchange mechanism 320. In general terms, the thermal energy exchange mechanism 320 causes a thermal energy exchange 303 between the pad 310 and the patient 180 and in turn, causes a thermal energy exchange 304 between the pad 310 and the environment 301.

The thermal energy exchange mechanism 320 includes multiple TEDs 330 embedded within a filing material 311 of the pad 310. Each of the TEDs 330 is coupled with the control module 186 via the electrical cable 194. The TEDs 330 can be dispersed horizontally along the pad 310. The TEDs 330 can also be dispersed vertically across the pad 310 between a top side 310A and a bottom side 310B of the pad 310. In some embodiments, one or more TEDs 330 can be disposed above one or more other TEDs 330. As such, the bottom sides 332 of one or more TEDs 330 can face the top sides 331 of one or more TEDs 330.

In some embodiments, the TEDs 330 can be arranged in layered arrays. For example, a top array can be positioned toward the top side 310A and a bottom array can be positioned toward the bottom side 310B. Other arrays can be disposed between the top array and the bottom array. Positioning the TEDs 330 vertically in relation to each other can enhance an efficiency of the TEDs 330 and/or enhance an efficiency of thermal energy transfer through the pad 310 as a whole.

The thermal exchange mechanism 320 includes one or more control temperature sensors such as control temperature sensors 340A, 340B, for example. The control temperature sensors 340A, 340B, are used to regulate the temperature of the top side 310A of the pad 310 as further described below. The thermal exchange mechanism 320 can also include one or more monitoring temperature sensors 341. The monitoring temperature sensors 341 can be used to keep the patient 180 safe from extreme temperatures in the case of a failure of the temperature control process in a manner similar to the monitoring temperature sensors 241 (FIG. 8).

The TEDs 330 are configured to define a temperature difference between the top side 331 and the bottom side 332 of each TED 330 in accordance with a direct-current (DC) voltage supplied to the TED 330. The magnitude of the temperature difference between the top side 331 and the bottom side 332 is defined by the magnitude of the DC voltage supplied to the TED 330 and direction of the temperature difference is defined by the polarity of the DC voltage. The polarity of the DC voltage as defined by the control module 186 can define a temperature of the top side 331 that is hotter than the bottom side 332. Conversely, a reversed polarity of the DC voltage as defined by the control module 186 can define a temperature of the top side 331 that is colder than the bottom side 332.

By way of example, the control module 186 can supply electrical energy to the TEDs 330 so that the temperature of the top side 310A is less than the temperature of the patient 180, and the temperature of the bottom side 310B is greater than the temperature of the environment 301. In this example, the thermal energy exchange 303 is directed away from the patient 180 toward the pad 310 thereby cooling the patient 180. Similarly, the thermal energy exchange 304 is directed away from the bottom side 310B of the pad 310 toward the environment 201.

By way of an alternative example, the control module 186 can supply electrical energy to the TEDs 330 so that the temperature of the top side 310A is greater than the temperature of the patient 180. In this example, the temperature of the bottom side 310B can be greater than or less than the temperature of the environment 301. In this example, the thermal energy exchange 303 is directed away from the pad 310 toward the patient 180 thereby warming the patient 180.

In some embodiments, the pad 310 can be divided into temperature zones, such as zones 320A, 320B, for example, so that portions of the patient 180 can be warmed or cooled differently. For example, the zone 320A can warm/cool the torso of the patient 180, and the zone 320B can warm/cool the head of the patient 180. Each zone can include one or more TEDs 330 and one or more control temperature sensors such as the control temperature sensors 340A, 340B. For example, in the illustrated embodiment, the zone 320A can include a subset of TEDs 330 together with the control temperature sensor 340A. Hence, electrical power can be supplied to the subset of TEDs 330 in accordance with regulating the temperature of zone 320A as measured by the control temperature sensor 340A. Similarly, the zone 320B can include another subset of TEDs 330 together with the control temperature sensor 340B, and electrical power can be supplied to the other subset of TEDs 330 in accordance with regulating the temperature of zone 320B as measured by the control temperature sensor 340B. In other embodiments, a temperature zone can include more than one control temperature sensor such as the control temperature sensor 340A or 340B. The temperature zones can be predefined or the zones can be dynamically configurable so that the clinician/operator can define the zones by assigning subsets of TEDs 330 to a zone upon each usage.

In some embodiments, the thermal exchange mechanism 320 can include an electric base 305 coupled with the electrical cable 194, and each of the TEDs 330, the control temperature sensors such as the control temperature sensors 340A, 340B, and/or the monitor temperature sensors 341 can be coupled with the electric base 305. The electric base 305 can be configured to receive electrical signals from the control temperature sensors 340A, 340B, and the monitor temperature sensors 341. The electric base 305 can further be configured distribute electrical power to the TEDs 330. In some embodiments, the electric base 305 can include electrical components (not shown) such as a processor, logic, and power converter to facilitate distribution of the electrical power to the TEDs 330. In use, the electric base 305 can receive electrical power and electrical signals from the control module 186, and the electric base 305 can also transmit electrical signals to the control module 186. The electric base 305 can then distribute electrical power (including polarity) to each of the TEDs 330 in accordance with electrical signals received from the control module 186. The electrical power supplied to a first TED can be different than an electrical power supplied to a second TED.

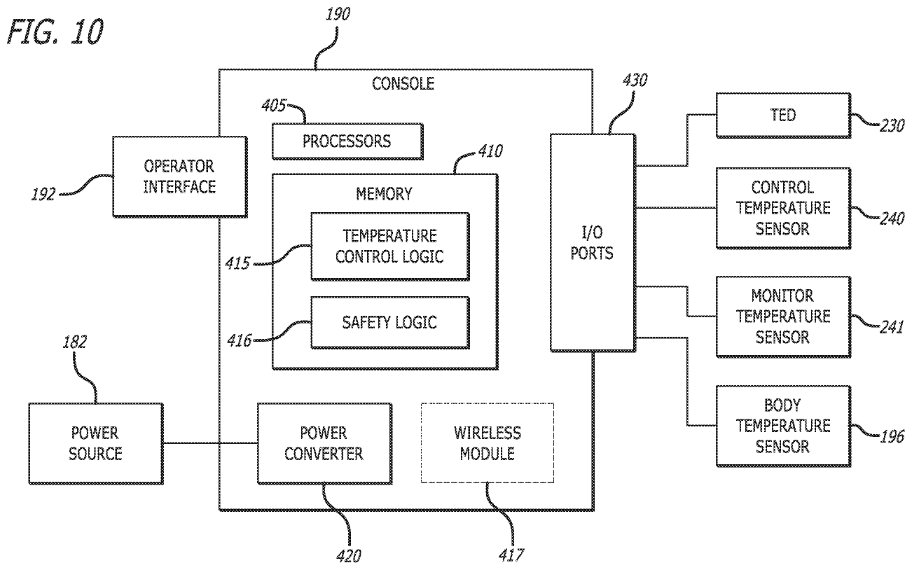

FIG. 10 illustrates a block diagram of the console 190 of FIG. 7, in accordance with some embodiments. The console 190 includes a one or more processors 405 and memory 410 including a non-transitory, computer-readable storage medium. Stored in the memory 410 are temperature control logic 415 and safety logic 416. The console 190 further includes a power converter 420, and input/output (I/O) ports 430. The console 190 receives electrical power from the power source 182. The I/O ports 430 facilitate connection of the console 190 with the TED 230, the control temperature sensor 240, the monitor temperature sensor 241, and the body temperature sensor 196. The console 190 can include or be coupled to the operator interface 192. In some embodiments, the console 190 can include a wireless module 417.

The power converter 420 converts electrical power from the power source 182 into a form of electrical power compatible with the TED 230. For example, the power source 182 can provide electrical power in the form of alternating current (AC) at a relatively high voltage (e.g., 120 to 240 VAC). The power converter 420 can convert the in-coming high AC voltage to a reduced DC voltage. In some embodiments, the DC voltage can be less than about 24 VDC to operate the TED 230. The power converter 420 can also be configured to reverse the polarity of the DC voltage. In other words, the power converter 420 can provide a DC voltage between about +24 VDC and about −24 VDC. In other embodiments, the DC voltage can be more or less than about 24 VDC to operate the TED 230. The power converter 420 can be controllable via the temperature control logic 415 when executed by the processor 405.

The operator interface 192 is coupled to the console 190 via a wired connection. In some embodiments, the operator interface 192 can be included with the console 190. In other embodiments, the operator interface 192 can be operated via an external device such as a network computer, a tablet or a cell phone via the wireless module 417.

The wireless module 417 can also provide for wireless connection to devices external of console 190. The external devices can include a facility network, the operator interface 192, the body temperature sensor 196, or any other device that can be used in accordance with operation of the system 184.

The temperature control logic 415 is configured to control the temperature of the top side 231 of the TED 230 (i.e., the temperature of the tray 225). The temperature control logic 415 can receive temperature data from the control temperature sensor 240 and adjust the DC voltage supplied to the TED 230 to move the temperature of the tray 225, toward a target bed temperature stored in the memory 410. More specifically, the temperature control logic 415 can compare temperature data received from the control temperature sensor 240 with the target bed temperature. As a result of the comparison, the temperature control logic 415 as executed by the one or more processors 405 can cause the power converter 420 to increase or decrease the DC voltage supplied to the TED 230 to move the bed temperature toward the target bed temperature. In some embodiments, adjusting the electrical power supplied to the TED includes switching a polarity of the electrical power. By way of summary, the temperature control logic 415 is configured to establish and maintain the bed temperature at the target bed temperature.

In some embodiments, the temperature control logic 415 can receive body temperature data from the body temperature sensor 196, and adjust the DC voltage supplied to the TED 230 to move the body temperature toward a target body temperature stored in the memory 410. More specifically, the temperature control logic 415 can compare temperature data received from the body temperature sensor 196 with the target body temperature. As a result of the comparison, the temperature control logic 415 can adjust the target bed temperature consistent with moving the body temperature toward the target body temperature stored in the memory 410.

The console 190 can include the safety logic 416 to prevent harm to the patient 180 in the case of a temperature control system failure. The safety logic 416 can compare temperature data received from the monitoring temperature sensor 241 with a high-temperature safety limit and/or a low-temperature safety limit stored in the memory 410. As a result of the comparison, the safety logic 416 as executed by the one or more processors 405 can cause the power converter 420 to adjust the DC voltage supplied to the TED 230. In some embodiments, the safety logic 416 can cause a complete interruption of the DC voltage supplied to the TED 230 and/or shut down operation of the system 184 completely to prevent the bed temperature from exceeding high-temperature safety limit and/or the low-temperature safety limit. Preventing the bed temperature from exceeding the high-temperature safety limit prevent hyperthermia of the patient 180 or causing a burn to the patient 180. Preventing the bed temperature from exceeding the low-temperature safety limit can prevent hypothermia of the patient 180.

In use, the clinician can preset a desired body temperature for the patient 180. The clinician can also preset a desired bed temperature. In some embodiments, the temperature control logic 415 can set the target bed temperature in accordance with the desired body temperature. The clinician can deform the pad 210 to define desired contours for the patient 180. The clinician can place the patient 180 on the pad 210 after which the pad 210 can maintain the contours to maintain the position of the patient.

### Methods

Methods of the TTM systems and pads set forth above include methods of use. For example, a method of using the system 100 includes a connecting step, a patient-placing step or patient-allowing step, and a therapeutic hypothermia-inducing step.

The method can further include a pad-placing step if the pad 104 is not already in place before the patient-placing step. The pad-placing step includes placing the pad 104 over a mattress of a hospital bed.

The connecting step includes connecting the one-or-more inlets 146 and the one-or-more outlets 148 of the conduit system 142 or 144 of the pad 104 to the hydraulic system 108 of the control module 102. If the pad 104 including the conduit system 142 is used, the connecting step can further include connecting a dedicated inlet for each conduit of the one-or-more conduits 152 to an outlet of the hydraulic system 108 of the control module 102, as well as connecting a dedicated outlet for each conduit of the one-or-more conduits 152 to an inlet of the hydraulic system 108 of the control module 102. Again, each conduit of the one-or-more conduits 152 is separated from other conduits of the one-or-more conduits 152 in the conduit system 142. However, if the pad 104 including the conduit system 144 is used, the connecting step includes connecting the dedicated inlet for the supply conduit 158 of the one-or more conduits 152 to an outlet of the hydraulic system 108 of the control module 102, as well as connecting the dedicated outlet for the supply conduit 158 to an inlet of the hydraulic system 108 of the control module 102. Again, the supply conduit 158 is configured to supply the primary conduit 154 and each successive conduit of the one-or-more expansion conduits 156 in parallel with the supply fluid in the conduit system 144.

The patient-placing step includes placing a patient on the pad 104, thereby allowing the pad body 140 of the pad 104 to conform to a body of the patient with the conformable material of the pad body 140. Similarly, the patient-allowing step includes allowing the patient to lie down on the pad 104, thereby allowing the pad body 140 of the pad 104 to conform to the body of the patient with the conformable material of the pad body 140.

The method can further include an adjusting step. The adjusting step includes adjusting a position of the patient on the pad 104 with respect to the primary conduit 154 and the one-or-more expansion conduits 156 of the one-or-more conduits 152 such that the patient is over the primary conduit 154 and a number of the one-or-more expansion conduits 156 sufficient to induce therapeutic hypothermia in the patient. If the pad 104 including the conduit system 144 is used, the adjusting step can further include adjusting the position of the patient over, for example, the pressure-activated valve of each conduit of the primary conduit and the number of one-or-more expansion conduits sufficient to induce therapeutic hypothermia in the patient.

The therapeutic hypothermia-inducing step includes inducing therapeutic hypothermia in the patient by circulating the supply fluid (e.g., a chilled fluid) provided by the control module 102 through the conduit system 142 or 144 of the pad 104.

Methods of the TTM systems and pads for neonates set forth above include a method of controlling a body temperature of the neonatal patient 180. The method includes (i) providing a neonatal bed 198, 300, the neonatal bed 198, 300 including the pad 210 having a thermally conductive filling material 211, (ii) deforming the pad 210 to define a contoured top surface of the pad 210 (iii) placing the neonatal patient 180 on the contoured top surface, and (iv) establishing a temperature of the pad 210, where establishing the temperature of the pad 210 includes suppling an electrical power to the thermal energy exchange mechanism 220 thermally coupled with the pad 210. In some embodiments, the thermal energy exchange mechanism 220 can include at least one thermoelectric device 230.

The method can further include adjusting the contoured top surface to enhance a thermal contact area between the pad 210 and the neonatal patient 180 as well as adjusting the contoured top surface to enhance a thermal contact area between the pad 210 and the neonatal patient 180, which can be performed after placing the neonatal patient 180 on the contoured top surface. The contoured top surface can inhibit movement of the neonatal patient 180 away from a patient position defined by the clinician.

The method can further include (i) measuring a body temperature of the neonatal patient 180, (ii) comparing the body temperature with a target body temperature, and establishing the temperature of the pad 210 to move the body temperature toward the target body temperature. Indeed, the method can further include (i) receiving a patient temperature signal from the patient temperature sensor 196, (ii) comparing the neonatal patient temperature signal with a target patient temperature stored in memory, and, as a result of the comparison, (iii) adjusting the target bed temperature. Relatedly, the method can further include (i) receiving by the control module 186 a control signal from the temperature sensor 240 coupled with the pad 210, (ii) comparing with logic stored in memory the control signal with a target bed temperature stored in the memory, and, as a result of the comparison, (iii) adjusting an electrical power supplied to the thermal energy exchange mechanism 220.

The method can also include comparing the pad 210 temperature with a safety limit and adjusting the electrical power supplied to the thermal energy exchange mechanism 220 to move the pad 210 temperature away from the high temperature limit. For example, the method can further include (i) receiving a safety signal from a safety temperature sensor coupled with the pad 210, (ii) comparing the safety signal with a safety temperature limit for the neonatal bed 198, 300 stored in the memory 410, and, as a result of the comparison, (iii) adjusting the electrical power supplied to the thermal energy exchange mechanism 220, which can include discontinuing the electrical power supplied to the thermal energy exchange mechanism 220. Indeed, the method can further include (i) receiving from the pad 210 temperature signal from a safety temperature sensor (e.g., the monitoring temperature sensors 241, 341), (ii) comparing the pad temperature signal with a high temperature limit, and (iii) adjusting the electrical power supplied to the thermal energy exchange mechanism 220 to move the pad temperature away from the high temperature limit. Similarly, the method can include comparing the pad temperature signal with a low temperature limit and adjusting the electrical power supplied to the thermal energy exchange mechanism 220 to move the pad temperature away from the low temperature limit.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures can be made from the particular embodiments disclosed herein without departing from the scope of the concepts provided herein.

What is claimed is:

1. A pad for targeted temperature management (“TTM”), comprising:
a pad body of a conformable material configured to conform to a body of a patient on the pad;
a conduit system disposed in the pad body or an overlayer configured for placement over the pad body, the conduit system including one or more conduits configured to convey a fluid through the pad body or the overlayer;
one or more inlets configured for charging the conduit system with the fluid; and
one or more outlets configured for discharging the fluid from the conduit system.

2. The pad of claim 1, wherein the one-or-more conduits include a primary conduit and one or more expansion conduits, each successive conduit of the one-or-more expansion conduits configured to accommodate a successively larger patient placed on the pad for TTM.

3. The pad of claim 2, wherein the primary conduit is located in a head-end portion of the pad body or the overlayer and each successive conduit of the one-or-more expansion conduits is located further toward a foot-end portion of the pad body or the overlayer.

4. The pad of claim 2, wherein each conduit of the one-or-more conduits is separated from other conduits of the one-or-more conduits, each conduit of the one-or-more conduits including a dedicated inlet of the one-or-more inlets and a dedicated outlet of the one-or-more outlets.

5. The pad of claim 2, wherein the one-or-more conduits include a supply conduit configured to supply the primary conduit and each successive conduit of the one-or-more expansion conduits in parallel with the fluid, the supply conduit including a dedicated inlet of the one-or-more inlets and a dedicated outlet of the one-or-more outlets.

6. The pad of claim 5, wherein the primary conduit and each successive conduit of the one-or-more expansion conduits includes a hand-actuated valve having a closed state and an open state.

7. The pad of claim 5, wherein the primary conduit and each successive conduit of the one-or-more expansion conduits includes a pressure-activated valve having a closed state and an open state, the valve configured to assume the open state by pressure applied thereto by the patient when the patient is placed or lies down on the pad over the valve.

8. The pad of claim 5, wherein the primary conduit and each successive conduit of the one-or-more expansion conduits includes an electrically activated valve having a closed state and an open state, the valve configured to assume the open state upon sensing the patient with an associated sensor when the patient is placed or lies down on the pad over the sensor.

9. The pad of claim 8, wherein the sensor is a tactile sensor or thermal photodetector configured to sense the patient physically touching the sensor when the patient is placed or lies down on the pad over the sensor.

10. The pad of claim 2, wherein the pad is configured for placement over a mattress of a hospital bed.

11. A system for targeted temperature management (“TTM”), comprising:
a control module including:
a chiller evaporator configured for chilling a fluid to produce a chilled fluid; and
a hydraulic system including one or more outlets configured for discharging the chilled fluid from the hydraulic system and one or more inlets configured for charging the hydraulic system with the fluid to continue to produce the chilled fluid; and
a pad including:
a pad body of a conformable material configured to conform to a body of a patient on the pad;
a conduit system disposed in the pad body or an overlayer configured for placement over the pad body, the conduit system including one or more conduits configured to convey the chilled fluid through the pad body or the overlayer;
one or more inlets configured for charging the conduit system with the chilled fluid; and
one or more outlets configured for discharging the fluid from the conduit system.

12. The system of claim 11, wherein the one-or-more conduits include a primary conduit and one or more expansion conduits, each successive conduit of the one-or-more expansion conduits configured to accommodate a successively larger patient placed on the pad for TTM.

13. The system of claim 12, wherein the primary conduit is located in a head-end portion of the pad body or the overlayer and each successive conduit of the one-or-more expansion conduits is located further toward a foot-end portion of the pad body or the overlayer.

14. The system of claim 12, wherein each conduit of the one-or-more conduits is separated from other conduits of the one-or-more conduits, each conduit of the one-or-more conduits including a dedicated inlet of the one-or-more inlets of the pad and a dedicated outlet of the one-or-more outlets of the pad.

15. The system of claim 12, wherein the one-or-more conduits include a supply conduit configured to supply the primary conduit and each successive conduit of the one-or-more expansion conduits in parallel with the fluid, the supply conduit including a dedicated inlet of the one-or-more inlets of the pad and a dedicated outlet of the one-or-more outlets of the pad.

16. The system of claim 15, wherein the primary conduit and each successive conduit of the one-or-more expansion conduits includes a hand-actuated valve having a closed state and an open state.

17. The system of claim 15, wherein the primary conduit and each successive conduit of the one-or-more expansion conduits includes a pressure-activated valve having a closed state and an open state, the valve configured to assume the open state by pressure applied thereto by the patient when the patient is placed or lies down on the pad over the valve.

18. The system of claim 15, wherein the primary conduit and each successive conduit of the one-or-more expansion conduits includes an electrically activated valve having a closed state and an open state, the valve configured to assume the open state upon sensing the patient with an associated sensor when the patient is placed or lies down on the pad over the sensor.

19. The system of claim 18, wherein the sensor is a tactile sensor or thermal photodetector configured to sense the patient physically touching the sensor when the patient is placed or lies down on the pad over the sensor.

20. The system of claim 12, wherein the pad is configured as a mattress of a hospital bed.

21. A system, comprising:
a neonatal bed configured for receiving a neonatal patient, the neonatal bed comprising a deformable pad; and
a thermal energy exchange mechanism coupled with the deformable pad,
wherein the thermal energy exchange mechanism is configured to extract thermal energy from the neonatal patient to cool the neonatal patient via a thermally conductive filling material of the deformable pad.

22. The system of claim 21, wherein the thermal energy exchange mechanism is configured to deliver thermal energy to the neonatal patient to warm the neonatal patient.

23. The system of claim 21, wherein the deformable pad is continuously deformable between a flat top surface and a contoured top surface, the contoured top surface including a formed depression configured to receive the neonatal patient thereon.

24. The system of claim 21, wherein the filling material has viscosity between about 10,000 centipoise and 1,000,000 centipoise.

25. The system of claim 21, wherein the filling material has a thermal conductivity greater than about 0.1 Watt/meter-° C.

26. The system of claim 21, wherein the filling material includes embedded objects to enhance the thermal conductivity of the filling material.

27. The system of claim 26, wherein the filling material has a thermal conductivity greater than about 2.0 Watt/meter-° C.

28. The system of claim 21, wherein the thermal energy exchange mechanism includes a thermoelectric device (TED).

29. The system of claim 28, wherein the neonatal bed includes a tray thermally coupled between the TED and the deformable pad, the tray configured to transfer thermal energy between the TED and the deformable pad.

30. The system of claim 29, wherein the tray is configured to laterally disperse thermal energy away from the TED to define a uniform temperature of the tray.
